(19) United States
(12) Patent Application Publication
(10) Pub. No.: US 2023/0138647 A1
(43) Pub. Date: May 4, 2023

(54) METHODS AND COMPOSITIONS FOR DETECTING VIRUSES

(71) Applicant: University of Louisville Research Foundation, Inc., Louisville, KY (US)

(72) Inventors: Joshua Fuqua, Pewee Valley, KY (US); Krystal Hamorsky, Crestwood, KY (US); Kenneth Palmer, Anchorage, KY (US); Jill M. Steinbach-Rankins, Louisville, KY (US)

(21) Appl. No.: 17/913,575

(22) PCT Filed: Mar. 25, 2021
(86) PCT No.: PCT/US2021/024219

§ 371 Date: Sep. 22, 2022

Related U.S. Application Data
(60) Provisional application No. 62/994,724, filed on Mar. 25, 2020.

(51) Int. Cl. G01N 33/543; G01N 33/569; G01N 33/577

(52) U.S. Cl. CPC G01N 33/54388; G01N 33/56983; G01N 33/54346; G01N 33/577; G01N 2333/4724; G01N 2469/10

(57) ABSTRACT

A lateral flow assay and compositions for performing such an assay are described herein that detect whole virus (e.g., HIV, HCV, HSV-2) or viral proteins that use the broad-spectrum anti-viral lectin, griffisthin (GRFT), conjugated to polymeric or gold nanoparticles (NPs) and an appropriate monoclonal antibody (mAb) to confer virus selectivity.

### CROSS REFERENCE TO RELATED APPLICATIONS

This application claims the benefit of priority under 35 U.S.C. 119(e) to U.S. Application No. 62/994,724 filed Mar. 25, 2020.

### TECHNICAL FIELD

This disclosure generally relates to methods and compositions for viral detection.

### BACKGROUND

The early and rapid diagnoses of infectious diseases, such as HIV, and timely initiation of appropriate treatments are critical determinants that promote positive clinical outcomes and general public health. Current HIV diagnostics are based on measures of viral load, CD4 cell count, and anti-HIV antibodies. The Centers for Disease Control and Prevention (CDC) step-wise algorithm for HIV diagnosis includes the HIV-1/2 antigen/antibody and antibody differentiation immunoassays, and nucleic acid testing (CDC Recommended Laboratory HIV Testing Algorithm for Serum or Plasma Specimens (2018)), all of which require specialized equipment and trained personnel.

These diagnostic approaches rely on viremia or serological events that detect analytes two to three weeks post-infection (FIG. 1). Only 50% of infected patients have detectable plasma RNA or p24 expression 12 days post-infection, resulting in only a fraction of cases being detected through ˜3 weeks post-infection. Critically, no diagnostic tools enable detection during the eclipse period (˜10 days post-infection). Furthermore, the conventional in vitro HIV RNA and antibody-based diagnostics are time-consuming and require centralized laboratories, experienced personnel, and bulky equipment.

Given this, the CDC and World Health Organization (WHO) have respectively recommended the development of improved methods, such as point-of-care (POC) or at-home testing, that can support the implementation of self-testing. Portable, user-friendly tests known for their simplicity, anonymity, and rapidity of detection have the potential to increase the likelihood that a patient will receive critical results more rapidly. While current FDA-approved POC tests have excellent performance, their sensitivity, in particular after recent infection, is inadequate relative to laboratory-based methods.

For example, the only FDA-approved “at-home” test for HIV works by detecting antibodies to HIV and thus does not capture the initial phase of viremia or viral rebound when HIV antibodies are in circulation. Here we propose to resolve this critical deficiency by developing a user-centered lateral flow test with the capability to detect whole virus through a finger stick during the acute stages of infection, to significantly inform treatment, improve patient prognosis, and prevent transmission of HIV as well as other glycosylated enveloped viruses.

### SUMMARY

This disclosure provides a modular protein-based platform that enables early, direct, and highly sensitive viral detection, while providing portability and ease of at-home use to significantly impact the prognoses, monitoring and treatment of viral infections and viral rebound. This innovative platform is exemplified herein using HIV, but can be readily adapted to detect (e.g., individually or simultaneously) any glycosylated enveloped virus (e.g, SARS-CoV-1, SARS-CoV-2, HCV, HSV-2, EBOLA, JEV, Nipah, Rabies) using the appropriate virus-specific antibodies (e.g., monoclonal antibodies).

In one aspect, an engineered GRFT polypeptide lacking lysines (“−K GRFT”) is provided.

In another aspect, an engineered GFRT polypeptide lacking lysines and having a M78K substitution is provided.

In yet another aspect, an engineered GRFT polypeptide lacking lysines and having a NK or CK substitution is provided.

In still another aspect, the engineered GRFT polypeptide described herein is conjugated to a nanoparticle. In some embodiments, the nanoparticle is a PLGA nanoparticle or a gold nanoparticle.

In another aspect, a solid substrate is provided that includes an engineered GRFT polypeptide as described herein. In some embodiments, the solid substrate is a lateral flow test strip. In some embodiments, the lateral flow test strip comprises cellulose, nitrocellulose, or combinations thereof.

In yet another aspect, an article of manufacture is provided for detecting the presence or absence of a virus. Such an article of manufacture can include an anti-virus antibody and an engineered GRFT polypeptide as described herein.

In some embodiments, the article of manufacture further includes a solid substrate. An exemplary solid substrate is a lateral flow test strip. In some embodiments, the anti-virus antibody is bound to the solid substrate; in some embodiments, an engineered GRFT polypeptide as described herein is bound to the solid substrate. In some embodiments, the assay is configured as a sandwich ELISA. In some embodiments, the anti-virus antibody is conjugated to a nanoparticle. Representative viruses that such an article of manufacture can be used to detect include, without limitation, HIV, SARS-CoV-1, SARS-CoV-2, HCV, HSV-2, EBOLA, JEV. Nipah, Rabies, and other glycosylated viruses.

In still another aspect, a method of detecting the presence or absence of a virus is provided. Such methods typically include contacting a solid substrate with a biological sample, wherein the solid substrate comprises an anti-virus antibody conjugated thereto to generate a capture complex; contacting the capture complex with an engineered GRFT polypeptide as described herein, to generate a detection complex; and detecting the detection complex.

In some embodiments, the virus is selected from HIV, SARS-CoV-1, SARS-CoV-2, HCV, HSV-2, EBOLA, JEV, Nipah, Rabies, and other glycosylated viruses. In some embodiments, the solid substrate is a lateral flow test strip. In some embodiments, the detecting step is performed within 30 mins of the contacting step in which a capture complex is generated. Representative biological samples include, without limitation, blood, saliva, urine, nasal secretions, feces, semen, or tears.

Unless otherwise defined, all technical and scientific terms used herein have the same meaning as commonly understood by one of ordinary skill in the art to which the methods and compositions of matter belong. Although methods and materials similar or equivalent to those described herein can be used in the practice or testing of the methods and compositions of matter, suitable methods and materials are described below. In addition, the materials, methods, and examples are illustrative only and not intended to be limiting. All publications, patent applications, patents, and other references mentioned herein are incorporated by reference in their entirety.

### DESCRIPTION OF DRAWINGS

FIG. 1 is a graph showing the viremia and serological events after infection detected using available diagnosis tools. Early viremia can be diagnosed through viral RNA or p24 antigen. Adapted from Hurt et al. (2017, Sex. Transm. Dis., 44:739-46).

FIG. 2 is a schematic showing a prototype virus sandwich assay using a combination of GRFT NPs and antibodies specific to the target virus assembled to the test line.

FIG. 3 is a graph showing the GRFT variants that were expressed and purified and had undergone selective lysine substitution. Subsequent amine coupling conjugation efficiency was assessed by fluorophore labeling.

FIG. 4 is a graph showing the activity of griffithsin variants conjugated to 2 kDa or 20 kDa mPEG polymers assessed via surface plasmon resonance. Two GRFT variants were identified that maintained pM affinity for the HIV glycoprotein gp120 when conjugated to two 20 kDa mPEGs.

FIG. 5 is a graph showing the anti-HIV-1 activity of GRFT variants against Env-pseudotyped Q769.h5 virus in HOS-CD4-CCR5+ cells. Percent neutralization was calculated by dividing the luminescence of sample wells by that of virus-only control wells. IC50s for each variant were determined by nonlinear regression analysis. The graphs, plotted in GraphPad Prism 5.0, are representative of two independent experiments each done in triplicate. Each data point represents the mean percentage inhibition f SEM of experimental triplicates.

FIG. 6 is a graph showing the results of an ELISA. VRC01 was coated on a plate and blocked with BSA, serial dilutions of gp120 (BAL) were added and incubated with −K GRFT M78K, and detected with anti-GRFT Abs. The various curves represent different combinations of VRC01 (1, 5, and 10 μg/mL) and −K GRFT M78K (0.5 and 5 μg/mL).

FIG. 7A is a schematic showing one type of surface-modification chemistry used to conjugate GRFT to polymers.

FIG. 7B is a graph showing antiviral activity of GRFT-modified fibers in TZM-bl cells upon exposure to decreasing concentrations of HIV-1, measured via luciferase activity. A dose-dependence was observed as a function of GRFT surface-modification and virus concentration. See, also, Grooms et al., 2016, Antimicrob. Agents Chemother., 60:6518-31.

FIG. 8 is a graph showing the SPR steady state response of GRFT-conjugated and unconjugated NPs for immobilized viral glycoprotein.

FIG. 9 is a graph showing VRC01 capture of HIV pseudovirus and GRFT detection.

### DETAILED DESCRIPTION

A first-in-class sandwich lateral flow assay is described herein that detects whole virus or viral proteins from glycosylated viruses (e.g., HIV, HCV, HSV-2, SARS-CoV-1, SARS-CoV-2, HCV, HSV-2, EBOLA, JEV, Nipah, Rabies) utilizing the broad-spectrum antiviral lectin, GRFT, conjugated to polymeric or gold nanoparticles (NPs) and an appropriate monoclonal antibody (mAb) to confer virus selectivity. The development of a protein-based diagnostic requires selective binding of an antigen in conjunction with the generation of a strong output signal, which features are met by the compositions and methods described herein. This disclosure provides a platform that addresses the urgent medical need for a broadly available tool to quickly diagnose viral infections to inform treatment and improve patient prognosis.

GRFT is a potent anti-viral lectin that binds mannose residues on viral envelopes and has demonstrated neutralizing activity against HIV, herpes simplex virus 2 (HSV-2), hepatitis C virus (HCV), Ebola, coronaviruses, and Nipahkf (Lusvarghi & Bewley, Griffithsin: An Antiviral Lectin with Outstanding Therapeutic Potential, Viruses, 8, 2016).

GRFT neutralizes a broad-spectrum of HIV strains at picomolar concentrations and is well tolerated in GLP-compliant toxicity studies. In addition, GRFT is highly stable with >2 years of room temperature stability and resistant to extremes in pH and temperature as well as to protease degradation (Moncla et al., 2011, Adv. Biosci. Biotechnol., 2:404-8). An oxidation resistant variant of GRFT, Q-GRFT, is currently in phase I clinical development as a viral prophylactic.

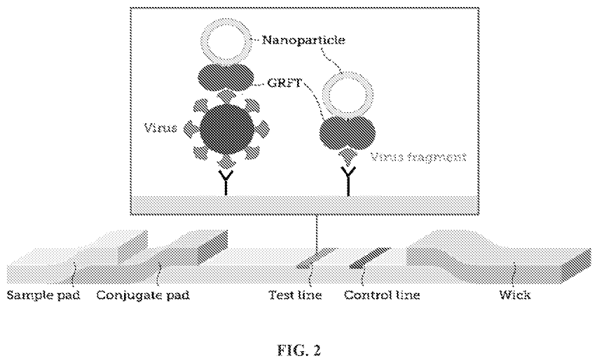

This document describes a novel approach that provides new avenues for the detection and identification of viruses that have glycans on their envelopes; the broad-spectrum binding activity of GRFT enables detection of essentially any glycosylated enveloped virus, with HIV detection being exemplified herein. In the proposed approach, GRFT will bind to virus and/or viral fragments, providing a visual output with polymeric and gold NPs (FIG. 2). Specificity, each targeted virus or viral protein is detected by a single mAb, imparting modularity. Therefore, by changing the mAb, multiple virus types may be distinguished individually or simultaneously.

NPs have been shown to impart optical and fluorescence properties that enable rapid and efficient clinical diagnostics (Draz & Shafiee, 2018, Theranostics, 8:1985-2017). In addition, NP probes have demonstrated advantages in terms of size, surface area, specificity, signal sensitivity, and stability, in addition to simple, rapid, highly-sensitive, label-free detection of numerous target molecules. Here, the known attributes of polymeric and gold NPs are combined with GRFT, which has demonstrated strong binding interactions with a number of different viruses (e.g., HIV, HCV, HSV-2), which has been specifically tailored to have improved stability and ease-of-conjugation. This is the first study investigating the use of multivalent GRFT NPs to maximize antigen coverage for rapid viral detection in a lateral flow device.

In addition to the epidemic posed by HIV-1 infection alone, five to ten percent of HIV-positive patients are co-infected with HCV, which is known to increase cardiovascular risk and mortality. Furthermore, HSV-2 and HIV co-infection are a known disease burden, which increases the risk of HIV infection. In addition to HIV. GRFT has anti-viral activity against both HCV and HSV-2, presenting a unique opportunity for dual diagnoses in co-infected patients. The specificity of the mAb component allows for modularity and expansion from HIV to one or more additional viruses with glycosylated envelopes. For example, HIV-specific mAbs to detect HIV-1 are described herein, however, the addition of mAbs specific to HCV and/or HSV-2 enables the tunability of the GRFT-based platform described herein to simultaneously diagnose co-infections.

The novel protein-based platform for viral diagnosis described herein can be readily deployed in an at-home device (e.g., a lateral flow test strip) that can provide rapid, patient-centered and cost-effective detection of early viral infection or relapse; enable earlier treatment; improve patient prognosis and decrease subsequent transmission.

### Polymeric and Gold Nanoparticles (NP) Conjugated to Lysine-Free (−K) GRFT

Two different nanoparticle (NP)-based lysine-free (−K) GRFT delivery platforms that can sensitively and specifically detect and amplify viral binding are described herein. PLGA and gold NPs with varying densities of GRFT can be generated to determine the effective concentration of GRFT on PLGA and gold NPs needed to achieve maximum binding to virions and/or one or more viral proteins.

(i) Determining the Density of −K GRFT to Saturate PLGA and Gold NP Surfaces

NP surface modification with −K GRFT: Carboxyl-terminated PLGA NPs can be synthesized as previously described (see, for example, Mahmoud et al., 2019, J. Control Release, 297:3-13) to allow for surface ligand addition during or after the fabrication process. Gold NPs and nanoshells (e.g., 150 nm), activated for amine conjugation, can be obtained commercially (e.g., nanoComposix) or produced using known methods (see, e.g., thermofisher.com/content/dam/LifeTech/images/integration/1602163_CrosslinkingHB_lores.pdf on the World Wide Web). Polymeric and gold NPs can be reacted using, for example, EDC-NHS chemistry in the presence of a range of −K GRFT concentrations, to determine the density at which PLGA and gold NP surfaces are saturated. The concentration of −K GRFT on the NP surface can be determined using complementary methods including ELISA quantification of −K GRFT; fluorescence spectroscopy; surface plasmon resonance (SPR); and/or size exclusion chromatography HPLC (see, e.g., Grooms et al., 2016, Antimicrob. Agents Chemother., 60:6518-31; Kramzer et al., 2021, AAPS PharmSciTech, 22:83; Fuqua et al., 2015, Plant Biotechnol., 13:1160-8).

To determine the amount and duration of surface-adsorbed versus covalently-bound GRFT, surface-mediated release of −K GRFT can be evaluated at fixed time points following incubation and quantified with ELISA. For physical characterization, un-hydrated NP morphology, diameter, and size distribution of PLGA and gold NPs can be evaluated using scanning or transmission electron microscopy, while dynamic light scattering and zeta potential analyses can be used to characterize the hydrodynamic diameter and surface charge of hydrated NPs.

(ii) Determining the Affinity/Avidity of −K GRMT-Modified NPs to Virion or Viral Proteins

The binding of −K GRFT-modified NPs to virions or one or more viral proteins (in the present examples, three gp120 proteins, one from HIV-1 clades A. B and C) can be assessed to determine the surface density needed to achieve maximum viral binding. The target goal is to achieve a sufficient number of virions or viral proteins adhered to −K GRFT NPs for detection to occur in a short amount of time (e.g., within 30 mins, 20 mins, 15 mins, 10 mins from the initial exposure to the virions or viral proteins). GRFT NP binding to virions or viral proteins can be determined by administering aliquots of PLGA or gold NPs formulated with different (low, medium and high) surface densities of −K GRFT to increasing amounts of virions or viral proteins (˜1 ng/mL to 1 mg/mL). −K GRFT-modified PLGA NPs can be synthesized to encapsulate a fluorescent dye (e.g., Coumarin 6) that remains within the NPs, enabling their visualization and quantification, while gold NPs have inherent optical absorbance properties. Unbound gp120 can be removed by centrifugation and the amount of bound NP can be determined by measuring residual fluorescence or optical absorbance, relative to unbound NPs remaining in the supernatant.

Binding affinity can be expressed as the fluorescence or absorbance of NPs bound for a given −K GRFT density, to a given concentration of virions or viral proteins. A non-glycosylated viral glycoprotein (e.g., made in E. coli) can serve as a control to assess non-specific binding, whereas unmodified NPs can be used to assess the specificity of −K GRFT NP adherence to the viral proteins. Binding experiments can be conducted in a variety of buffer solutions and sera-containing media. Similar groups can be used in Surface Plasmon Resonance (SPR) experiments to determine the Kₒₙ/K_{off} rates and KD for surface-modified NP and virion/viral protein interactions. NP preparations that exhibit strong binding to virions and viral proteins can be tested for their ability to bind to viral-specific mAbs, and undergo transport in a lateral flow assay. Statistical analysis between groups can be determined using one-way ANOVA (p<0.05).

### Identifying Anti-Viral Antibodies Capable of Capturing Viral Glycoproteins and Pseudovirions in the Presence of GR-T and GRFT NPs

One important advantage of an immuno-chromatographic test is the specificity afforded by antigen-antibody interactions. Monoclonal Abs against the desired virus or viral protein can be screened with ELISA and/or Surface Plasmon Resonance (SPR) format(s) to determine the antibodies that maximize the breadth and selectivity of this platform. In addition, pseudoviruses can be used for screening breadth and selectivity of mAbs to virions in the presence of GRFT. At least one (e.g., at least two, at least three) antibodies can be selected for the capture of virions and viral proteins for lateral flow prototype development. Antibodies typically are selected based on low cross-reactivity, high affinity binding and lack of interference with −K GRFT/−K GRFT NP binding.

(i) Screening Monoclonal Antibodies that Capture or Detect Viral Proteins in the Presence of −K GRFT and −K GRFT NPs

mAbs to virions or viral proteins can be purchased or produced. Methods of producing mAbs are known in the art. For example, the full-length IgG mAbs can be expressed in N. benthamiana using the magnlCON vector as described herein (e.g., for VRC01, a mAb against HIV) and purified by FPLC using Protein A and an additional chromatography step (e.g., ceramic hydroxyapatite), if needed.

With respect to methods and compositions for detecting HIV, there are ten anti-HIV mAbs (Table 1; see, also, Dashti et al., 2019, Trends Mol. Med., 25:228-40) that have higher % neutralization breadth than VRC01, which provides >88% coverage of HIV strains. Eight of the mAbs are specific to the CD4 binding site, and two of the mAbs are specific to the gp41 epitope; therefore, these mAbs are not expected to interfere with −K GRFT binding. As described herein, these ten HIV Abs can be screened to assess affinity, breadth, and specificity to HIV.

TABLE 1
Exemplary Anti-HIV Monoclonal Antibodies

| mAb | Epitope | % breadth |
|---|---|---|
| N49P6 | CD4bs | 100 |
| N49P7 | CD4bs | 100 |
| N49P11 | CD4bs | 100 |
| N6 | CD4bs | 98 |
| VRC07 | CD4bs | 92 |
| 12A12 | CD4bs | 92 |
| 3BNC117 | CD4bs | 90 |
| N49P9 | CD4bs | 89 |
| VRC01 | CD4bs | 88 |
| DH511-2 | gp41 | 98 |
| 10E8 | gp41 | 97 |

CD4-specific mAbs can be tested in ELISA format to ensure that the mAb and the GRFT can synergize. The mAbs and −K GRFT or −K GRFT-modified NPs can be analyzed. For example, mAbs diluted in PBS can be coated on a microtiter plate, the plates can be blocked, the virions or viral proteins can be added in serial dilutions, followed by incubation with −K GRFT and −K GRFT NPs. Bound −K GRFT and −K GRFT NPs can be detected with anti-GRFT antibodies. ELISA methods can be modified from the traditional 1-hour incubation to a 15 minute incubation with the virions or viral proteins, which is more amenable to a lateral flow design.

CD4-specific mAbs capable of binding virions or viral proteins in the presence of GRFT or GRFT NPs can be further screened in the presence of pseudovirus. In the same format, pseudovirus can be sandwiched with mAb and GRFT to select mAbs that bind when GRFT present. Pseudoviruses are the primary screening mechanism for gp41 epitope specific mAbs. The criteria for mAb selection is ≤20% change in potency of the mAb with and without −K GRFT or −K GRFT NPs.

Surface Plasmon Resonance (SPR) also can be used to determine the kinetic parameters of mAb to virions or viral proteins and −K GRFT and −K GRFT NPs to virions or viral proteins captured with mAb. Each mAb protein can be captured on a sensor chip via an anti-human IgG (Fc) antibody of IgG1 isotype, and various concentrations of recombinant virions or viral proteins can be used as analytes. Each mAb assay can be performed in triplicate. The curves can be fit based on 1:1 binding kinetics to determine kinetic parameters. In addition, virions or viral proteins/−K GRFT and virions or viral proteins/−K GRFT-NP complexes can be injected as analyte to determine kinetic parameters of the complexes to mAb. Complexes that do not disrupt the binding affinity of mAb to respective virions or viral proteins more than 20% and have fast association kinetics can be selected as potential candidates.

(ii) Determining mAb and −K GRFT NP Characteristics in the Presence of Interferants and Biological Fluids

All selected mAbs can be screened by sandwich ELISA (mAb as capture; GRFT as detection) in biological fluids spiked with potentially interfering viral proteins (e.g., in the case of HIV, HCV and HSV-2 antigens) to assess the impact of these interferents. For each viral protein, four dose curves can be designed (e.g., one in phosphate buffered saline (PBS), one in 90% plasma, one in 90% plasma plus 1 μg/mL glycosylated HCV E2 antigen, and one in 90% plasma plus 1 μg/mL glycosylated HSV-2 antigen). E. coli-produced viral proteins can be used as a negative control. Analysis can be done in triplicate, and half-maximal effective concentration (EC₅₀) values can be compared using ANOVA in GraphPad Prism. To provide robustness and further feasibility, co-incubation of −K GRFT NPs with viral proteins can be analyzed. Curves can be designed as above, except −K GRFT and −K GRFT NPs can be incubated with serial dilutions of viral proteins prior to incubation with mAb. These steps then can be repeated using pseudovirus in place of the viral proteins. Acceptance criteria for selective mAb selection can be mAbs with less than 2% change in binding in the presence of plasma and <20% reduction in binding to viral proteins in the presence of interfering proteins.

Lateral flow assays have the ability to disrupt interfering plasma antibodies by a pH shift, buffer composition, or surfactants. Therefore, the impact of anti-viral antibodies in plasma can be assessed by spiking plasma with polyclonal Abs to the target virus. Using ELISA format, for each viral protein, four dose curves can be designed (e.g., one in phosphate buffered saline (PBS), one in 90% plasma, one in 90% plasma plus polyclonal anti-viral proteins, and one in 90% plasma plus polyclonal anti-viral proteins) shifted to pH 2.0. E. coli-produced viral proteins can be used as a negative control. ELISA methods can be modified if the presence of a polyclonal antibody saturates binding and, therefore, inhibits GRFT and mAb binding. Analysis can be done in triplicate and half-maximal effective concentration (EC₅₀) values can be compared using ANOVA in GraphPad Prism.

Assessing GRFT NPs and mAbs in a Lateral Flow Format

−K GRFT NPs and mAbs capable of capturing the viral proteins and the pseudovirus are integrated into a nitrocellulose-based lateral flow assay. Immobilized Ab candidates and preliminary −K GRFT NP formulations can be used to identify lateral flow baseline conditions. Strips with the selected mAbs and polymeric and gold NPs that maximize binding to viral proteins and pseudoviruses (or attenuated viruses) can be prototyped.

(i) Choosing Affinity Reagents

mAbs can be conjugated to NPs and dispensed onto a nitrocellulose membrane as a test line. For each target, 3 different pseudoviruses and viral antigens can be evaluated. −K GRFT NPs can be dispensed onto nitrocellulose membranes, and mAb (i.e., conjugate and test line) can be tested against the complementary −K GRFT NPs to build an analyte capture sandwich for the whole virus or viral fragments (see, for example. FIG. 2). A standard prototype lateral flow test strip can be assembled that includes, for example, a glass fiber sample pad for low volume retention, a slow wicking nitrocellulose for maximum sensitivity, and cellulose absorbent pad. −K GRFT NPs in solution can be mixed with pseudovirus or purified viral proteins prior to application to the lateral flow test strip. The relative intensity of the test line can be qualitatively assessed by eye, as well as measured quantitatively via colorimetric change. Antibody/−K GRFT NP combinations that provide the strongest test line signal in the presence of analyte spiked into buffer without non-specific binding in negative samples can be selected for further optimization.

(ii) Specificity

To evaluate the specificity of the assays to the target analyte (e.g., the virion or viral proteins), the optimal polymeric and gold −K GRFT NPs, and antibodies that have the highest specific signal when tested with contrived samples, can be tested against common interfering antibodies and known co-circulating viruses. Testing can be performed with contrived samples prepared with other virus clades or using virus-positive patient samples that have been adjudicated prior to testing in accordance with the current medical practice. Pairs that have little to no signal in the presence of cross-reactive species can be chosen for further optimization.

(iii) Optimization

The initial focus of optimization can be the conditions for conjugation of the mAb or −K GRFT to the NP. The ratio of mAb or −K GRFT to NP, NP blocking reagent, incubation times, and other reaction parameters can be evaluated to improve conjugate performance. At this stage, testing can transition from contrived samples prepared by spiking analyte into buffer to analyte spiked into representative sample matrix. Materials for each test strip component can be evaluated, and the associated chemistries optimized (e.g., sample pad pre-treatment) to allow for consistent normalization of the sample matrix. The top running conditions can be selected based on sensitivity and specificity. After a set of running conditions has been selected using wet −K GRFT NPs, the process of drying down the −K GRFT NPs onto the sample pad can be optimized. A range of chemistries can be tested to obtain the best NP release from the conjugate pad upon resuspension with sample. In addition, sample volume, running buffer, and assay run time can be examined.

(iv) Preliminary Production of a Lateral Flow Test Strip

Once a functional dried conjugate pad is obtained, a set of test strips (e.g., 2×100 test strips) can be generated for further evaluation. The functionality and specificity of lateral flow devices can be tested with pseudoviruses and viral proteins diluted in blood and in the presence of other glycosylated viral proteins.

### Refining and Optimizing the Functionality of GRFT-Based Lateral Flow Assays

After the initial lateral flow assay (LFA) assay has been designed, a variety of factors can be optimized to achieve maximal detection capability. For example:

Stability: One important factor that can be optimized is the stability of the polymeric or gold NP formulations in solution and at different storage conditions, as these are governing parameters that will impact flow and sensitivity of detection in the lateral flow device. First, the stability of different NP formulations can be evaluated to reduce aggregation and ensure mono-dispersity, particularly in high salt conditions. While maximum conjugation density is evaluated, the amount of mAb/GRFT surface modification can be altered to reduce aggregation, while ensuring a sufficient modification density of mAb/GRFT to cover and stabilize the NPs. One benefit of reducing modification density is reducing production costs and decreasing non-specific binding. Additionally, using the optimal mAb identified, NP formulations can be made with different conjugation strategies, e.g., citrate conjugation for subsequent thiol reactivity, avidin-biotin, or PEGylation, to increase stability, minimize steric hindrance, or to link conjugates. Additionally, while nanoComposix has established that 150 nm carboxyl gold nanoshells exhibit high sensitivity in rapid diagnostic tests, the use of 40 or 80 nm carboxyl gold nanoparticles may serve to increase stability, reduce Ab costs, and enable more reproducible conjugates.

Nanoparticle formulations produced with these variations can be assessed with UV-vis spectrophotometry, DLS, and zeta potential measurements similar to those described herein, and can be characterized as a function of pH, minimum mAb conjugation, impact of the addition of stabilizing agents (such as different concentrations of BSA, PEG, etc.) and other conjugation strategies. Additionally, selected gold and polymeric NP formulations can be assessed for stability over a duration of time (e.g., 6 months, 9 months, 1 yr) under similar conditions and as a function of storage temperature (room temperature (RT), 4° C., and −20° C.) and humidity (0, 40, 65, 90%).

Physical Flow within the LFA: To optimize NP flow, it may be necessary to reduce adhesion to the conjugate pad or minimize the likelihood of NP agglomeration (i.e., clogging) on the pad. In this case, conjugation conditions can be re-optimized to enhance stability as discussed herein. Additionally, if the sample does not reach the conjugate pad, smaller particles can be selected and validated in each group. This can be achieved using enhanced centrifugation and filtration measurements for polymeric NPs, and filtration for gold NPs. Additionally, as mentioned herein, gold NPs can be purchased within the size range of 40, 80, or 100 nm, or nanoshells of 120 nm, to alleviate transport through the lateral flow assay. Lastly, different concentrations of NPs can be administered to the pad, as dilution factor is known to impact flow and detection capabilities (lesser with less dilute samples), in addition to cost.

Increasing Binding Signal: In line with the required optimization of nanoparticle flow through the lateral flow device, utilizing gold or polymeric nanoparticles or nanoshell configurations help to establish the best binding characteristics, flow through characteristics and resulting differences in binding signal. Hence, information learned herein can be used to vary bioreceptor conjugation density for these different groups to maximize binding signal.

Specificity-Sensitivity: As detection is required in biologically-relevant and complex environments (e.g., human blood), non-specific binding of GRFT NPs or mAb NPs can be addressed by fully characterizing modification density as a ratio of surface coverage, adding or increasing the amount of BSA or PEG, or considering different conjugation strategies that may contribute to charge and hydrophobicity characteristics that, in turn, may contribute to non-specific binding. In these cases. NP characteristics can be further refined to achieve the highest specificity levels in the lateral flow assay. In cases in which sensitivity is inadequate and cannot differentiate two close concentration values, the NP size, type, modification density and design can be changed to maximize sensitivity.

Control and Test Line Optimization: A variety of factors may contribute to inadequate visualization of control and/or test lines, suggesting that the mAb NP and GRFT NP conjugation can be optimized for stability or binding affinity. Here, the conjugation strategy or conditions can change. Furthermore, if the control line is hand to visualize but the test line is apparent, the bioreceptor-NP conjugate concentration can be adjusted or increased. Alternatively, a different NP can be used that is specific for the control line or the NP can be changed to a different capture bioreceptor.

Expansion of Detection Capabilities: In addition to virions and/or primary viral proteins, it may be desirable to rapidly detect a secondary viral protein from the relevant virus. For example, in HIV-1, gp41 enables a secondary method of detection that can be used alone or in combination with gp120 detection to enhance detection capabilities. Similarly, the ability to detect NPs in more complex environments presented by blood or saliva is integral to enabling sensitive and specific detection. Datasets with different particle groups in healthy patient blood samples can be used to verify, validate, and compare variation across groups.

### Verifying and Validating Lateral Flow Test Strips

A viable lateral flow product needs to demonstrate repeatable and reproducible results while differentiating negative and positive responses with high diagnostic sensitivity and specificity. GRFT-lateral flow test strips can be manufactured in bulk to be tested for diagnostic sensitivity and specificity in human biological fluids in the presence of other interferents.

Multiple factors can be assessed with a cohort of positive and negative samples to determine the overall viability of the lateral flow test strips and the ability to attain reproducible results. Multiple large-scale prototype manufacturing runs can be completed to allow for refinement of the design and continued improvement of lateral flow performance.

For example, 500 lateral flow test strips as described herein can be manufactured for testing. Testing can be performed as outlined below for diagnostic sensitivity, specificity and precision.

(i) Diagnostic Sensitivity and Specificity

A cohort of viral-negative and viral-positive patient samples can be obtained and/or generated to test sensitivity, specificity, and precision of each design. Simply by way of example, a negative cohort can have 40 HIV negative sera including at least 5 HCV and 5 HSV-2 positive samples, and a positive cohort can have 40 HIV positive sera stratified by controlled viral load at <200 copies/mL, rebounding HIV positive at 200-1000 copies/mL, and undiagnosed HIV positive at 1000-100,000 copies/mL. The positive cohort can contain at least 5 sera that are HCV or HSV-2 positive. The viral load in the positive samples can be confirmed using RT-PCR. The target should have less than 10% false positives or false negatives.

(ii) Repeatability and Reproducibility

The procedure for diagnostic sensitivity and specificity outlined herein can be repeated a total of three times by one technician to determine the repeatability. A second technician in a different laboratory also can run the samples a total of 3 times. The six runs can be combined to determine the precision between laboratories. The collaborative precision can be determined by variance in positive and negative agreement between the 6-individual tests.

In accordance with the present invention, there may be employed conventional molecular biology, microbiology, biochemical, and recombinant DNA techniques within the skill of the art. Such techniques are explained fully in the literature. The invention will be further described in the following examples, which do not limit the scope of the methods and compositions of matter described in the claims.

### EXAMPLES

A novel and broadly-applicable approach is described herein that may be diversified to diagnose many glycosylated enveloped viruses. Given that GRFT is a stable broad-spectrum lectin, any glycosylated enveloped virus that binds to GRFT may be detected using this platform, and the methods and compositions described herein can be tuned to detect specific panels of glycosylated viruses for clinical application. While the Examples below are focused on a platform that enables the detection of HIV whole virus, this platform can be used to generate a new family of protein-based diagnostic tools that allow for detection of numerous viruses in a rapid, user-centered, sensitive, accurate, selective, and inexpensive manner. This description is the first step towards developing an at-home technology for detection of early infection, enabling informed treatment, thereby improving patient prognosis and subsequent transmission risk.

### Example 1—GRFT was Structurally Optimized for Amine Coupling to Enhance Efficiency and Maintain Activity

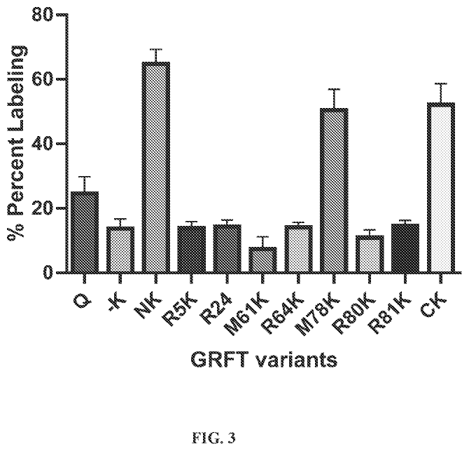

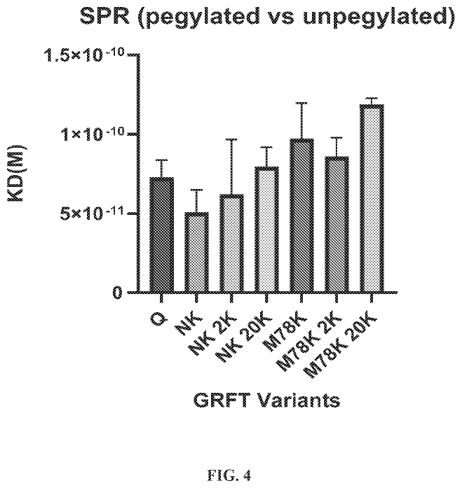

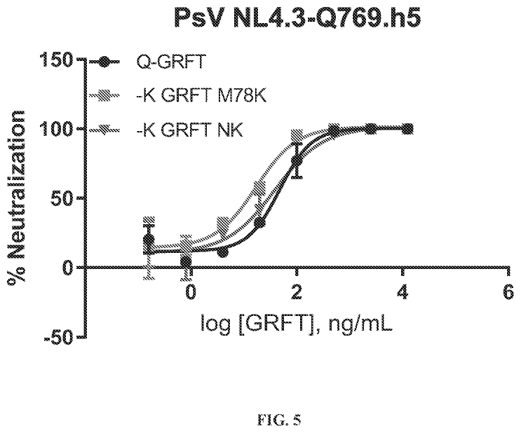

Wild type GRFT and Q-GRFT (M78Q) both demonstrate poor conjugation through amine coupling reactions due to a scarcity of free lysines. To address this, lysines were removed from the Q-GRFT amino acid structure (at amino acid positions 6 and 99), single lysines were systematically substituted back into GRFT at every arginine (amino acid positions 5, 24, 64, 80, or 81), methionine (amino acid positions 61 or 78), or at each termini (N- or C-), and variants were assessed for labeling efficiency (FIG. 3) and activity (FIG. 4) when conjugated to 2 or 20 kDa methoxypolyethylene glycol (mPEG) polymers. Based on the variants assessed, −K GRFT M78K and −K GRFT NK (collectively referred to as −K GRFT) were selected for further characterization due to their enhanced labeling efficiency and high affinity to gp120. The Kₒₙ and K_{off} rates for −K GRFT M78K were 7.18×10⁶ M⁻¹s⁻¹ and 6.23×10⁻⁴ s⁻¹, respectively. Similarly, −K NK had a Kₒₙ rate of 1.04×10⁷ M⁻¹s⁻¹ and a K_{off} rate of 6.83×10⁴ s⁻¹. Both variants had similar HIV neutralization capacity (FIG. 5), demonstrating that the modifications do not impact GRFT potency. These −K GRFT variants were used for NP modifications.

### Example 2—−K GRFT M78K can Detect Gp120 Bound to HIV-Specific Monoclonal Antibody

To impart specificity to this diagnostic, a mAb specific to HIV was used. VRC01 is a CD4 binding site-specific broadly neutralizing mAb isolated from an HIV-1-infected donor, that has demonstrated safe and tolerable infusions in a randomized clinical trial (Riddler et al., 2018, Open Forum Infect. Dis., 5:ofy242). VRC01 has neutralization coverage of ˜90% genetically diverse heterologous HIV-1 (Wu et al., 2010. Science, 329:856-61) and HIV-2 strains (Kumar et al., 2017, Front Immunol., 8:1568). A new production system was developed for VRC01 in Nicotiana benthamiana plants using a single tobamovirus replicon vector. Plant-made VRC01 exhibited HIV neutralization synergism with GRFT (Hamorsky et al., 2013, Antimicrob. Agents Chemother., 57:2076-86) due to exposure of the CD4 binding site, by GRFT-HIV gp120 binding (Alexandre et al., 2011, J. Virol., 85:9039-50). Thus, VRC01 was used during this development as a base line control in assays.

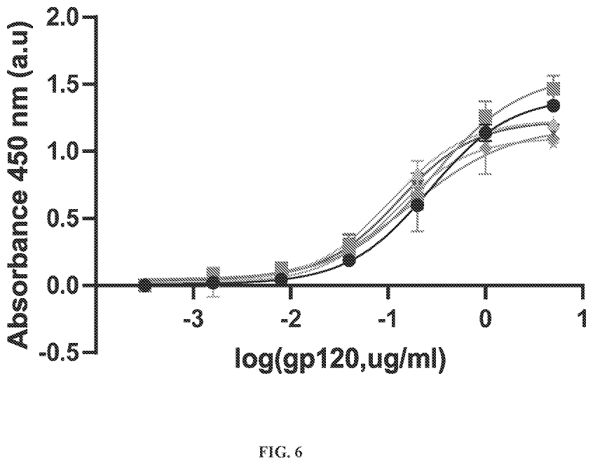

Using an enzyme-linked immunosorbent assay (ELISA) (FIG. 6), different concentrations of VRC01 and −K GRFT M78K, respectively, were used to capture and detect gp120 (Bal-1). These experiments demonstrate that GRFT and VRC01 both bind to gp120 simultaneously, in a dose-dependent manner (EC₅₀˜0.2 μg/mL) and with a preliminary lower detection limit of 1 ng/mL gp120. Taken together, this data provide strong rationale to use VRC01 as an initial mAb for internal standard, due to its complementary binding, specificity to HIV, and broad-spectrum binding to different HIV clades and strains.

### Example 3—GRFT- and Other Protein-Modified Delivery Vehicles Potently Inhibit Viral and Bacterial Infections

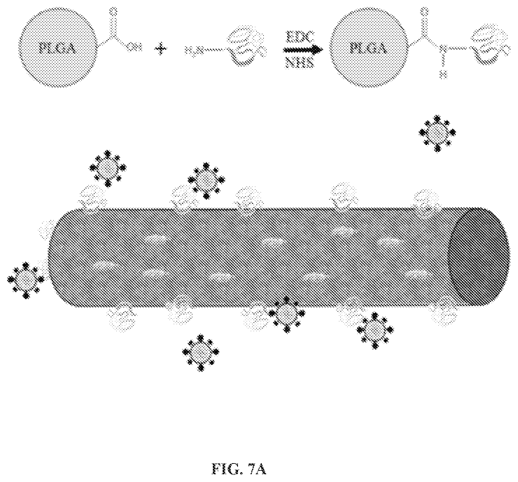

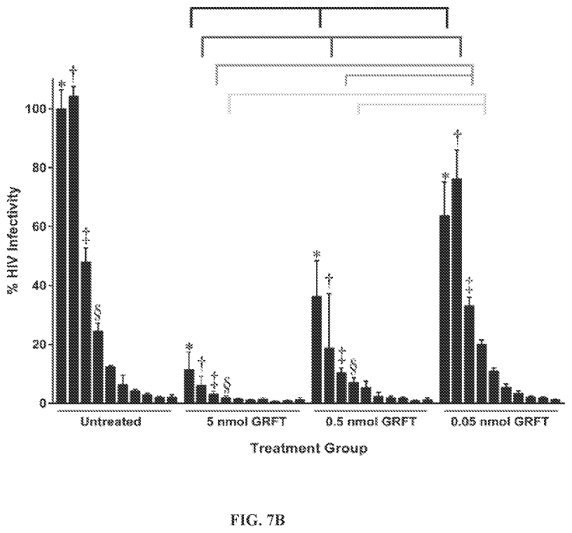

Our previous work has developed polymeric nanoparticles and fibers that are surface-modified with GRFT or other proteins, to inhibit HIV and HSV-2 infections or bacterial infections, respectively. Similar modification schemes with GRFT (FIG. 7A) as proposed here, demonstrate strong anti-HIV activity as a function of GRFT-modification density and virus concentration (FIG. 7B). Similar surface modification strategies also were used to design NPs modified with proteins or peptides that adhere to other pathogens. Mechanistic binding and in vivo efficacy studies demonstrated a significant increase in NP potency, relative to free peptide, due to multivalency of peptides on the NP surface. These results further demonstrate the capability of GRFT-modified vehicles to bind to and immobilize viral and bacterial pathogens.

### Example 4—Activity of −K GRFT M78K Conjugated to NPs

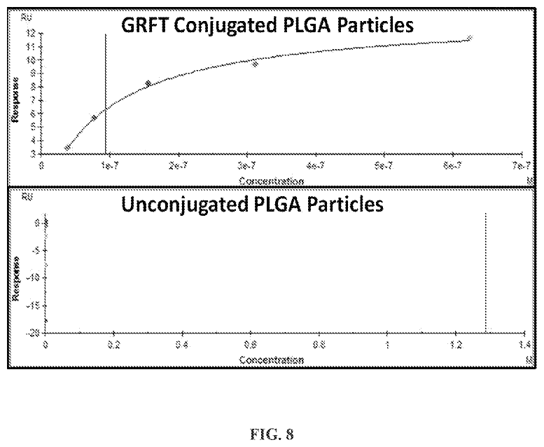

As proof-of-concept, PLGA NPs were conjugated to −K GRFT M78K, and preliminary results demonstrated that GRFT-modified NPs had the capacity to bind to gp120 and other viral glycoproteins. When assessed by surface plasmon resonance (SPR), −K GRFT M78K NPs were responsive to immobilized SARS-CoV-2 spike glycoprotein, while unconjugated NPs exhibited no binding interactions (FIG. 8). Thus GRFT-modified NPs are active against enveloped viral proteins and viral selectivity will be imparted by mAbs to specific viruses.

### Example 5—VRC01 Capture of HIV Pseudovirus and Detection with GRFT

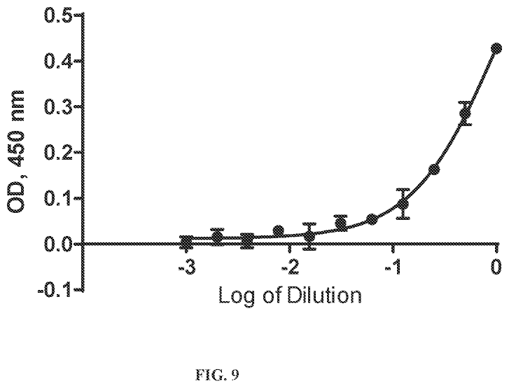

An 96 well microtiter plate was coated with monoclonal antibody, VRC01, at 10 μg/mL. HIV pseudovirus Q769.h5 was added to column 1 undiluted, titrated 2-fold across the plate in Dulbecco's Modified Eagle Medium (Gibco 10569-010), and then incubated at 37° C. for 1 hour. Biotinylated-K-Q-Griffithsin was added at 10 μg/mL and detected using Streptavidin-HRP at 1:15000. OD values were blank subtracted. Log(agonist) vs. response—Variable slope (four parameters) in GraphPad Prism was used to fit the curve. Data from these experiments indicate the ability to capture pseudovirus in a sandwich format using a mAb to capture and GRFT to detect. See FIG. 9.

### Example 6—Experimental Rigor and Robustness

All assays were replicated and appropriately powered to allow data analysis by suitable statistical methods (e.g., t-tests, ANOVA with Bonferroni's post-hoc tests, etc.).

Significance was established with a p-value <0.05.

### Example 7—Sequence of GRFT

(SEQ ID NO: 1)
SLTHRKFGGS GGSPFSGLSS IAVRSGSYLD XIIIDGVHHG
GSGGNLSPTE TFGSGEYISN MTIRSGDYID NISFETNMGR
RFGPYGGSGG SANTLSNVKV IQINGSAGDY LDSLDIYYEQ Y

### Example 8—Griffithsin-Based Microbial Detection

See Appendix A

It is to be understood that, while the methods and compositions of matter have been described herein in conjunction with a number of different aspects, the foregoing description of the various aspects is intended to illustrate and not limit the scope of the methods and compositions of matter. Other aspects, advantages, and modifications are within the scope of the following claims.

Disclosed are methods and compositions that can be used for, can be used in conjunction with, can be used in preparation for, or are products of the disclosed methods and compositions. These and other materials are disclosed herein, and it is understood that combinations, subsets, interactions, groups, etc. of these methods and compositions are disclosed. That is, while specific reference to each various individual and collective combinations and permutations of these compositions and methods may not be explicitly disclosed, each is specifically contemplated and described herein. For example, if a particular composition of matter or a particular method is disclosed and discussed and a number of compositions or methods are discussed, each and every combination and permutation of the compositions and the methods are specifically contemplated unless specifically indicated to the contrary. Likewise, any subset or combination of these is also specifically contemplated and disclosed.

### APPENDIX A

### Part A

Griffithsin (GRFT) is a lectin with antiviral activity. It can bind to the envelope of glycosylated viruses and inhibit their activity. Currently, it is under development for both prophylactic and therapeutic applications of multiple viruses.

Griffithsin has the potential to be used in diagnostic platforms.

### Part B

Early accurate diagnosis of HIV is critical for preventing transmission and improving treatment. HIV is diagnosed by HIV viral load test, CD4 cell count, and anti-HIV antibodies. The CDC algorithm for HIV testing is HIV1/2 antigen/antibody immunoassay, HIV1/2 antibody differentiation immunoassay and nucleic acid testing (REF). These laboratory assays require specialized equipment and trained personnel. Modern rapid point of care (POC) test have been developed and are FDA approved, however they are less sensitive than traditional methods. Direct detection of HIV virus is a promising method for rapid, real-time, and sensitive identification of HIV infection (REF). According to the World Health Organization (WHO), POC tests that address infectious disease control, especially for the developing countries, should follow “ASSURED” criteria: (1) affordable, (2) sensitive, (3) specific, (4) user-friendly, (5) rapid and robust, (6) equipment-free and (7) deliverable to end-users (REF). There is a great medical need for POC technologies meeting ASSURED criteria for diagnosis of HIV infection.

GRFT is a lectin that binds mannose residues on the HIV gp120 envelope spike protein with nanomolar affinity and has the capacity to neutralize HIV-1 at picomolar concentrations [1, 2]. GRFT has broad neutralizing activity against sexually co-transmitted viruses including HSV-2 and HCV [3, 4] and is minimally cytotoxic [5-8]. One GRFT variant, engineered for resistance to oxidation (M78Q, Q-GRFT), has undergone rigorous preclinical efficacy and safety evaluations. Investigators at the University of Louisville, who are now members of GROW Biomedicine, LLC, have recently guided Q-GRFT to a first-in-human Phase I clinical study as an enema-based topical prophylactic within the NIAID-supported PREVENT U19 Program Project.

Our long term goal is to develop a Q-GRFT-based POC test that meets ASSURED criteria and is capable of early diagnosis of HIV infection. Towards obtaining our long term goal we plan to develop an immunochromatographic test with the following objectives: (i) determine the optimal GRFT—colorimetric enzyme conjugate, (ii) elucidate the specificity of an immunochromatographic test with various anti-HIV antibodies, and (iii) test analytical parameters of the immunochromatographic test. We hypothesize that enzyme conjugated Q-GRFT will be a platform to directly detect HIV pseudo virus bound to a monoclonal anti-HIV antibody. Preliminary data generated has shown. This data presents great rationale for determining the feasibility of a Q-GRFT based immunochromatographic test towards simple, specific, rapid and sensitive determination of HIV infection. Three specific aims are proposed.

Aim 1: Colorimetric enzyme conjugated to Q-GRFT. Based on preliminary data, HRP can be conjugated to a unique lysine variant of Q-GRFT and the molecule maintains gp120 binding activity. The biological recognition event occurring in a biosensing platform must contain a detection modality: optical, electrical, mechanic, etc. Optical detection is known for its ability to be used in quick lateral flow POC devices. The working hypotheses is an optimized HRP-Q-GRFT will be utilized as the biosensing component of an immunochromatographic test.

Aim 2: Identify an anti-HIV antibody for capable of capturing gp120 bound to HRP-Q-GRFT. One important advantage of an immunochromatographic test the specificity afforded by antigen antibody interactions. Discovering the correct antibody Q-GRFT pair afforded a selective system.

Aim 3: Evaluate the analytical parameters of the immunochromatographic test. Feasibility towards ASSURED criteria will be determined by measure the breadth gp120 binding and sensitivity. Demonstrating broad gp120 binding and a highly sensitive system provides rational for further development.

If successful, this research will make available novel diagnostic platform for HIV diagnosis. It is envisioned that Q-GRFT will be employed in POC device for rapid, simple and cost-effective detection of early infection. These sensing systems will address the urgent medical need for a broadly available tool to quickly diagnosis virus infections. Early and accurate diagnosis will inform treatment therefore improving patient prognosis. Furthermore this research opens up new avenues to utilize the developed platform for other glycosylated enveloped viruses such as HCV and coronaviruses as well as multiplex analysis.

### Part C

Coronaviruses are significant health concerns, with three distinct pandemic threats, SARS, MERS, and SARS-2, emerging since 2003. Additionally, there are at least four endemic coronaviruses, HKU1, OC43, NL63, and 229E, that carry an increased mortality risk when they progress to viral pneumonia. All coronavirus screening currently uses a PCR based assay that can delay patient care and clinical risk management. We are proposing the development of a screening tool to allow point-of-care diagnosis of coronaviruses with the expectation that there will be therapies developed from the current epidemic that will allow treatment of coronaviruses in the near term. We are proposing an assay system that detects viral particles in the blood or oropharyngeal secretions. Selectivity for viral particles would be imparted through immobilized monoclonal antibodies and detection would be accomplished by coating viral particles with the lectin, griffithsin, that is conjugated to a reporter molecule. We are screening reporters that provide the best sensitivity and the least binding interference and are considering, fluorometric, nanoparticle, and colorimetric reporters.

We will conjugate griffithsin to enzymes that are active against colorimetric substrates such as horseradish peroxidase and screen for binding affinity to coronavirus proteins. Additionally, antibodies will be screened for selectivity against viral proteins. Once the detection assay is optimized, the student will detect inactivated coronavirus with the detection system.

We will utilize many protein expression, purification, and analytical techniques including, protein gels, FPLC, HPLC, ELISA, and Surface Plasmon Resonance. We will collect data demonstrating the feasibility of coronavirus particle detection with the proposed platform, which should provide the evidence necessary to develop a clinically focused prototype.

### Part D

Coronaviruses are significant health concerns, with three distinct pandemic threats, SARS-CoV-1, MERS, and SARS-CoV-2, emerging since 2003. In addition, there are at least four endemic coronaviruses, HKU1, OC43, NL63, and 229E, that carry an increased mortality risk when they progress to viral pneumonia. All coronavirus screening currently uses real-time polymerase chain reaction (RT-PCR)-based assays that can delay patient care and clinical risk management. Early and accurate diagnoses of coronaviruses is critical for preventing transmission and enabling treatment. According to the World Health Organization (WHO), point of care (POC) tests that address infectious disease control, especially for developing countries, should aim for “ASSURED” criteria in that they are: (1) affordable, (2) sensitive, (3) specific, (4) user-friendly, (5) rapid and robust, (6) equipment-free and (7) deliverable to end-users. Due to shortcomings in both the availability and rapidity of current testing methods, there is an urgent medical need for POC technologies meeting ASSURED criteria to diagnose and potentially enable timely treatment of coronavirus infections. Moreover, flexible and broadly acting POC assays may be easily adapted to address future emergent coronaviruses.

Here we propose the development of a nanoparticle (NP)-based lateral flow device to provide POC diagnosis of coronaviruses, in blood or oropharyngeal secretions. Selectivity for viral particles in patient secretions will be imparted through immobilized monoclonal antibodies and detection will be accomplished by binding viral particles with the lectin, Griffithsin (GRFT), that is conjugated to a NP reporter molecule. The following tasks will culminate in a GRFT-NP-based lateral flow assay prototype, toward the long-term vision of developing a pan-coronavirus POC diagnostic:

Task 1. Develop polymeric nanoparticles, that are surface-modified with GRFT, as the basis for a lateral flow POC assay.

Task 2. Identify anti-coronavirus antibodies capable of capturing spike proteins bound to GRFT nanoparticles.

Task 3. Develop a sandwich format lateral flow assay, based on GRFT nanoparticles, that is optimized for the target analytes of both SARS-CoV-1 and SARS-CoV-2.

Preliminary Data: Griffithsin Activity and Nanoparticle Conjugation

GRFT is a lectin that binds mannose residues on viral envelopes and has demonstrated potent neutralizing activity against coronaviruses, HIV, Ebola, HSV-2, and Nipah. GRFT is highly stable and resistant to pH, temperature, and protease degradation, making it a unique protein to act as a bioreceptor in a POC diagnostic¹. Extensive work in our groups has focused on the design and development of a variety of NP and fiber-based platforms, incorporating the antiviral protein GRFT, that we propose may be employed for the detection of coronaviruses.

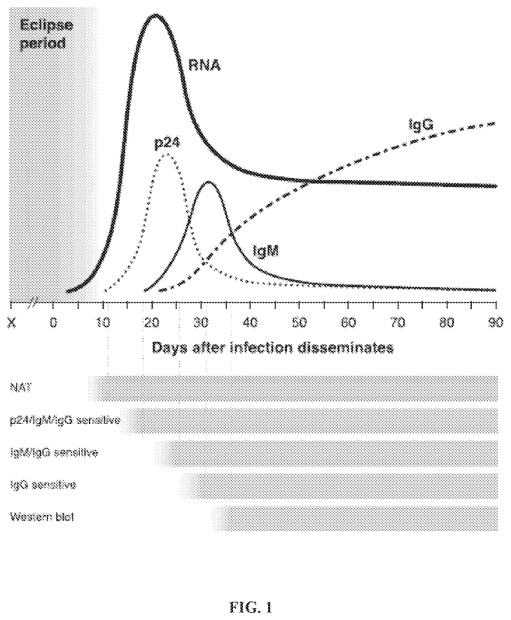

GRFT binds to coronaviruses, including SARS-CoV-1, MERS, and SARS-CoV-2. In animal models of SARS-CoV, GRFT has demonstrated prophylactic activity and neutralizing affinity to SARS, MERS, and endemic viruses². We have assessed the affinity of GRFT for MERS, SARS-CoV-1, and SARS-CoV-2 spike proteins using surface plasmon resonance (SPR) (FIG. 1). GRFT had the highest affinity for MERS and SARS-CoV-2 spike protein, but had nanomolar affinity for all coronavirus spike proteins, including SARS-CoV-1.

GRFT has been structurally optimized for amine coupling to enhance efficiency and maintain activity. Wild-type GRFT demonstrates poor conjugation through amine coupling reactions due to a scarcity of free lysines. To address this, we removed all lysines from the GRFT amino acid structure, systematically substituted single lysines back into the molecule, and assessed efficiency and activity when conjugated to a 20 kDa methoxypolyethylene glycol (mPEG) polymer. We identified three variants, −K GRFT M78K, −K GRFT NK, and −K GRFT CK (collectively referred to as −kGRFT), that maintain activity when conjugated to large polymers and select these for subsequent NP modification. As proof-of-concept, poly(lactic-co-glycolic acid) (PLGA) NPs were conjugated to −K GRFT M78K, and preliminary results demonstrate that GRFT-modified NPs have the capacity to bind to SARS-CoV-2 spike protein. When assessed by SPR, GRFT-NPs were responsive to immobilized SARS-CoV-2 spike glycoprotein, while unconjugated NPs exhibited no binding interactions (FIG. 2).

In other work, we have demonstrated the ability to incorporate GRFT in a variety of delivery vehicle formulations to meet different environmental and temporal delivery needs. We have fabricated pH-responsive mPEG-poly(lactic-co-glycolic acid):poly (butylacrylate-co-acrylic acid) (mPEG-PLGA:PBA-co-PAA) fibers that release GRFT upon exposure to different environmental pH. In other work, we formulated GRFT PLGA and mPEG-PLGA NPs³, that demonstrate high GRFT loading efficiency (e.g., 70% or 70 μg GRFT/mg NP). The most highly loaded mPEG-PLGA NPs were subsequently evaluated against HIV-1 infection in vitro and demonstrated similar inhibition to free GRFT (FIG. 3). Expanding upon these formulations, we recently integrated GRFT NPs in single-layered hydrophobic (PLGA or polycaprolactone (PCL)) and multilayered PCL-polyethylene oxide (PEO)-PCL fibers. Nanoparticle-fiber composites demonstrated prolonged GRFT release for up to 90 d and in vivo efficacy against HSV-2 infection³.

Together these data highlight our groups' expertise in protein engineering and delivery vehicle development, lending strong support to the expansion of GRFT and multivalent surface-modified GRFT-NPs as a platform diagnostic tool. While preliminary experiments demonstrate that GRFT and its variants bind coronavirus viral spike proteins with high affinity, binding avidity and signal intensity are enhanced by multivalent GRFT conjugation to reporter NPs. Based on these preliminary data, we hypothesize that GRFT-NPs can be used as a platform to bind coronaviruses and sensitively detect virus. To our knowledge, this is the first study to explore the potential of GRFT and GRFT-NPs as a new rapid screening strategy for coronaviruses and our initial proof-of concept will focus on distinguishing SARS-CoV-1 and SARS-CoV-2, with the long term goal of developing a pan-coronavirus POC device.

### Tasks and Deliverables

Task 1. Develop polymeric nanoparticles, that are surface-modified with GRFT, as the basis for a lateral flow assay. Here we seek to utilize our expertise in the delivery of GRFT, to explore a nanoparticle-based −kGRFT delivery platform that can potently detect and amplify coronavirus spike protein binding. We will first synthesize NPs with varying concentrations of −kGRFT to determine the input concentration of −kGRFT needed to saturate and obtain functional densities of −kGRFT on the NP surface. The concentration of GRFT on the NP surface will be determined using complementary methods that include ELISA quantification of −kGRFT; fluorescence spectroscopy; SPR; and size exclusion chromatography HPLC. For −kGRFT-NP-coronavirus spike binding affinity studies, low, medium, and high-density (valency) NP formulations will be assessed for the ability to bind to a range of coronavirus concentrations.

Deliverable: Identify the formulation that provides the highest level of SARS-CoV-1 and/or SARS-CoV-2 binding and detection, relative to unconjugated NPs.

Task 2. Identify anti-coronavirus antibodies capable of capturing spike proteins bound to kGRFT nanoparticles. One important advantage of an immunochromatographic test is the specificity afforded by antigen-antibody interactions. Available monoclonal coronavirus antibodies will be screened with ELISA and/or SPR format(s) to determine the antibod(ies) that maximize(s) the selectivity of this platform. The selected antibod(ies) will be subsequently used to coat microtiter plates or immobilize on gold chips. SARS-CoV-1 and SARS-CoV-2 spike proteins will be added at various concentrations and detected with −kGRFT-NPs.

Deliverables: Select at least one antibody for the capture of both SARS-CoV-1 and SARS-CoV-2 spike proteins. The antibodies will be chosen based on cross-reactivity, high affinity binding and lack of interference with −kGRFT-NP binding.

Task 3; Develop a sandwich format lateral flow assay, based on −kGRFT nanoparticles, that is optimized for the target analyte of SARS-CoV-2. Free −kGRFT and kGRFT-NPs will be integrated in the design of a cellulose-based lateral flow assay, that incorporates identified antibodies capable of capturing the SARS-CoV-2 spike protein. We will work with a sub-contractor that specializes in the design and prototyping of lateral flow assays for rapid diagnostic applications. Briefly, physical parameters including strip geometry, paper porosity, and lateral flow volume and rate as a function of NP size and specimen type/aliquot will be optimized. In addition, antibody immobilization methods, NP stability, antibody cross-reactivity, and antibody/NP concentrations will be optimized to maximize sensitivity (detecting CoV-2 when present), while maintaining specificity (avoiding a false positive). During Y1, we will work with free −kGRFT, immobilized Ab candidates, and preliminary NP formulations to identify a baseline for testing. In Y2, this device will be prototyped for antibody binding to free CoV-2, −kGRFT-NPs bound to CoV-2, and CoV-2-kGRFT-NP-Ab binding for detection.

Deliverable: Prototype GRFT-NP lateral flow device for detecting SARS-CoV-2.

### Novelty and Impact

This research will make available a novel diagnostic platform for coronavirus diagnosis. It is envisioned that −kGRFT will be employed in a POC device for rapid, simple and cost-effective detection of early infection that will inform treatment, thereby improving patient prognosis. Furthermore, this work is the first step toward developing a POC technology to multiplex various viruses in the clinical setting and can easily be tuned by utilizing different capture antibodies to target emerging novel coronaviruses.

1. An engineered GRFT polypeptide lacking lysines (“−K GRFT”).

2. An engineered GFRT polypeptide lacking lysines and having a M78K substitution.

3. An engineered GRFT polypeptide lacking lysines and having a NK or CK substitution.

4. The engineered GRFT polypeptide of claim 1, conjugated to a nanoparticle.

5. The engineered GRFT polypeptide of claim 4, wherein the nanoparticle is a PLGA nanoparticle or a gold nanoparticle.

6. A solid substrate comprising the engineered GRFT polypeptide of claim 1.

7. The solid substrate of claim 6, wherein the solid substrate is a lateral flow test strip.

8. The solid substrate of claim 7, wherein the lateral flow test strip comprises cellulose, nitrocellulose, or combinations thereof.

9. An article of manufacture for detecting the presence or absence of a virus, comprising an anti-virus antibody and the engineered GRFT polypeptide of claim 1.

10. The article of manufacture of claim 9, further comprising a solid substrate.

11. The article of manufacture of claim 10, wherein the solid substrate is a lateral flow test strip.

12. The article of manufacture of claim 10, wherein the anti-virus antibody is bound to the solid substrate.

13. The article of manufacture of claim 10, wherein the engineered GRFT polypeptide is bound to the solid substrate.

14. The article of manufacture of claim 9, wherein the assay is configured as a sandwich ELISA.

15. The article of manufacture of claim 9, wherein the anti-virus antibody is conjugated to a nanoparticle.

16. The article of manufacture of claim 9, wherein the virus is selected from HIV, SARS-CoV-1, SARS-CoV-2, HCV, HSV-2, EBOLA, JEV, Nipah, and Rabies.

17. A method of detecting the presence or absence of a virus, comprising:
contacting a solid substrate with a biological sample, wherein the solid substrate comprises an anti-virus antibody conjugated thereto to generate a capture complex;
contacting the capture complex with the engineered GRFT polypeptide of claim 1, to generate a detection complex; and
detecting the detection complex.

18. The method of claim 17, wherein the virus is selected from HIV, SARS-CoV-1, SARS-CoV-2, HCV, HSV-2, EBOLA, JEV, Nipah, and Rabies.

19. The method of claim 17, wherein the solid substrate is a lateral flow test strip.

20. The method of claim 17, wherein the detecting step is performed within 30 mins of the contacting step in which a capture complex is generated.

21. The method of claim 17, wherein the biological sample is blood, saliva, urine, nasal secretions, feces, semen, or tears.


SEQUENCE LISTING (1 sequence)

SEQ ID NO: 1 - protein, 121 aa, Griffithsia sp.
  misc_feature (31)..(31): Xaa can be any naturally occurring amino acid
SLTHRKFGGSGGSPFSGLSSIAVRSGSYLDXIIIDGVHHGGSGGNLSPTFTFGSGEYISNMTIRSGDYIDNISFETNMGRRFGPYGGSGGSANTLSNVKVIQINGSAGDYLDSLDIYYEQY